(19) United States
(12) Patent Application Publication
(10) Pub. No.: US 2010/0089394 A1
(43) Pub. Date: Apr. 15, 2010

(54) INHALER AND DRIVING METHOD FOR SAME

(75) Inventors: Naoko Sakurada, Yokohama-shi (JP); Masaru Sugita, Tokyo (JP); Hideki Kaneko, Yokohama-shi (JP); Yohei Masada, Kawasaki-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/598,121

(22) PCT Filed: May 30, 2008
(86) PCT No.: PCT/JP2008/060403

§ 371 Date: Oct. 29, 2009

(30) Foreign Application Priority Data
Jun. 5, 2007 (JP) 2007-148844

(51) Int. Cl. A61M 15/00

(52) U.S. Cl. 128/203.14

(57) ABSTRACT

The present invention provides an inhaler capable of measuring lung function, feeding back the measurements, and administering medicine suitable for a lung condition in a single use. The inhaler 1 of the present invention includes a lung function measuring unit 2 having a spirometer for measuring lung function, and, using a computing unit 5, compares the lung function measurements with a medication pattern table stored in a storage unit 7 and determines a disease severity and a diseased region of the lung. Based on the determination results, the computing unit further selects types and particle diameters of medicines suitable for the lung condition, and controls an ejection unit 3 including a plurality of ejection portions to eject the medicines required for treatment in a single use.

### TECHNICAL FIELD

The present invention relates to an inhaler which measures lung function, determines a medicine type and particle diameter from the measurements, and ejects a medicine suitable for the lung condition.

### BACKGROUND ART

Well-known apparatuses for measuring lung function include spirometers and peak flow meters. Doctors determine the severity of lung diseases such as bronchial asthma and chronic obstructive pulmonary disease (COPD) from FEV1 (forced expiratory volume in one second) values or PEF (peak expiratory flow) values obtained from the measurements, and select a type and amount of medicine to be administered. From the pattern of a flow volume curve recording changes in flow (speed, flow) and volume at maximum effort during expiration, it is possible to make judgments about the occluded region of the lungs.

Inhalers representative of those used in medicine include metered dose inhalers (MDI), dry powder inhalers (DPI) and nebulizers (see U.S. Pat. No. 5,542,410 and US Patent Application No. 2003/0098022). Methods, other than those using the above-described inhalers, for forming liquid sample into minute droplets and ejecting the droplets include well-known ink jet techniques.

The above-described medicine ejection apparatuses include not only apparatuses which eject a single material, but also apparatuses which eject materials of a plurality of types. Such apparatuses are variously used to eject a medicine and an adjuvant or a plurality of medicines. Besides medicines, the apparatuses are used to eject a wide range of materials, such as compounds for use in treatments, flavorings, or colorants.

In pharmacotherapy for lung diseases such as bronchial asthma and chronic pulmonary obstructive disease (COPD), the drugs principally employed are bronchodilators and inhaled glucocorticosteroids. It is recommended that appropriate medicine is administered in amounts which vary in steps according to the severity of the case. To determine the disease severity lung function tests are essential and are preferably performed continuously. However, since such tests are largely performed in medical facilities, it is difficult to do a test and apply feedback from the test to every administration of the medicine. Moreover, to achieve more effective treatment by efficiently administering the medicine to the diseased region of the lungs, medicine with a particle diameter suitable for the condition of the lungs can be ejected. For patient usability, the medicines of the plurality of types and particle diameters necessary for the treatment can be ejected in a single use.

However, there is currently no inhaler capable of determining the condition of the lungs from lung function measurements, selecting types and particle diameters of medicine based on the results, and ejecting the medicine of a plurality of types and particle diameters in a single use. U.S. Pat. No. 5,542,410 records an aerosolized medicine delivery device which measures inspiratory flow, and determines a particle dimension distribution and dose of an aerosolized compound. However, U.S. Pat. No. 5,542,410 does not disclose a means of automatically selecting the type of medicine based on the respiratory flow measurements. Moreover, the device is not capable of simultaneously discharging medicines of differing particle dimensions.

US Patent Application No. 2003/0098022 discloses a peak flow meter (spirometer) for measuring respiratory function in patients and a nebulizer which is an inhaler for administering medicine. However, the peak flow meter is not capable of automatically determining a type and particle diameter of the medicine to be ejected based on the respiratory function measurements.

### DISCLOSURE OF THE INVENTION

The present invention provides an inhaler and a driving method thereof capable of measuring lung function using a spirometer and administering, in a single use, the necessary medicine using feedback from the measurements.

In order to solve the above-described problems, the inhaler of the present invention for causing the user to inhale an ejected medicine includes:

a lung function measuring unit operable to measure lung function;

a computing unit operable to find a medicine type and particle diameter suitable for a disease severity and a diseased region determined from measurements by the lung function measuring unit; and

an ejection unit operable to eject the medicine based on output from the computing unit.

The inhaler of the present invention enables medicines of types and amounts dependent on the disease severity to be automatically administered to the precise region of the lungs. Hence, it is possible to efficiently administer the medicine and to reduce error in the administered amounts and types of the medicine.

Since the required medicine can be ejected in a single use, there is no need to switch cartridges and inhale a number of times, and the load on the user is therefore light.

Further, by measuring lung function before and after each administration of the medicine and simultaneously recording and managing the lung function measurements and treatment records, it may be possible to implement early treatment and prevent acute exacerbation. Since it is possible to measure lung function and administer the medicine using a single apparatus, lung conditions can be managed continuously and in a manner which is very convenient for the user.

Other features and advantages of the present invention will be apparent from the following description taken in conjunction with the accompanying drawings, in which like reference characters designate the same or similar portions throughout the figures thereof.

### BRIEF DESCRIPTION OF THE DRAWINGS

FIG. 1 is a block diagram illustrating an inhaler of a first embodiment.

FIG. 2 is a perspective drawing illustrating a medicine cartridge of the inhaler of FIG. 1.

FIG. 3 is a perspective drawing illustrating an appearance of the inhaler of FIG. 1.

FIG. 4 is a perspective drawing illustrating an access cover of the inhaler of FIG. 3 in an open state.

FIG. 5 is comprised of FIGS. 5A and 5B are a flowchart illustrating a flow of operations in the inhaler.

FIG. 6 is a table illustrating measurements by the lung function measuring unit and corresponding medication contents according to examples 1 to 12.

FIG. 7 is a table illustrating measurements by the lung function measuring unit and corresponding medication contents according to examples 13 to 21.

FIG. 8 is a table illustrating measurements by the lung function measuring unit and corresponding medication contents according to examples 22 to 30.

### BEST MODE FOR CARRYING OUT THE INVENTION

Preferred embodiments of the present invention will now be described in detail in accordance with the accompanying drawings.

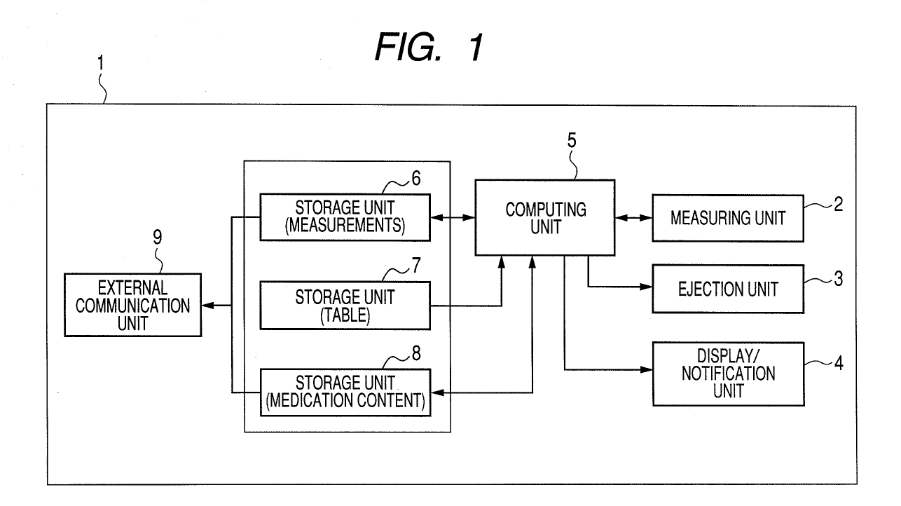

The inhaler 1 illustrated in FIG. 1 includes a lung function measuring unit 2 including a sensor such as a spirometer, a medicine ejection unit 3 including a driving unit, a display/notification unit 4, a computing unit 5 for selecting medicine types and particle diameters based on the disease severity and the diseased region determined from the lung function measurement results. The inhaler 1 further includes a storage unit 6 for storing lung function measurements, a storage unit (determining unit) 7 having a medication pattern table used for determining lung condition and selecting the medicines to be ejected, a storage unit 8 for storing medication content (recording unit), and an external communication unit 9.

The display/notification unit 4 displays a process from the lung function measurement to the medicine ejection, and warns the user when, after continued administration, a number of administered doses has reached a limit without an improvement of lung function.

The computing unit 5 compares the lung function measurements for the user obtained from the lung function measuring unit 2 with the medication pattern table (table) stored in the storage unit 7, determines types and particle diameters of the medicine to be ejected, and outputs the results. Based on the results (output), the ejection unit 3 is controlled to eject medicine of the required types and particle diameters. An arrangement in which the computing unit 5 determines the amounts of medicine to be ejected as well as the types and particle diameters is also possible. In other words, by comparing the measurements of the lung function of the user obtained from the lung function measuring unit 2 and the medication pattern table (table) stored in the storage unit 7, it is possible to determine the required types, amounts, and particle diameters of the medicine.

The external communication unit 9 communicates, by a commonly used communication method, with the health management center or hospital that is supporting the user. The communication includes transmission of the lung function measurements, and recording of the administered medication. The external communication unit 9 is also capable of transmitting data required to determine the types and amounts of medicine and of obtaining the consent of the doctor when the medication content is to change.

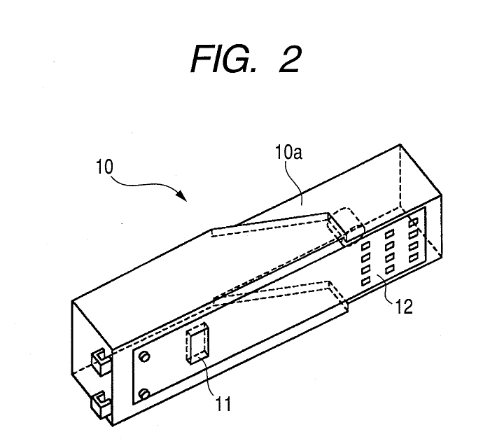

FIG. 2 is a perspective drawing illustrating the medicine cartridge (cartridge) 10 which forms the ejection unit 3 of the inhaler 1. The medicine cartridge 10 is manufactured to form, on a same substrate, a single body from a head portion (ejection head) 11 for ejecting the medicine, a reservoir 10a for storing the medicine, and a flow path for passing the medicine from the reservoir 10a to the head portion 11. A controller for controlling the driving of the head portion 11 exchanges driving signals, control signals with the head portion 11 via an electrical connection portion 12 to which internal wiring is connected. There are no particular limitations on the construction of the ejection unit 3. The reservoir and the ejection head may be formed into single body so as to form a cartridge as shown in FIG. 2. Alternatively, the reservoir and the ejection head may be constructed as separate bodies.

In the present invention, the head portion 11 includes ejection energy generation elements which may be of a desired type. Examples of ejection energy generation elements include electrothermal conversion elements which confer heat energy to the medicine and electromechanical conversion elements which confer mechanical energy to the medicine. That is, methods for ejecting the medicine include a method (thermal jet method) by which the medicine is ejected from an ejection openings by conferring heat energy to the medicine using the electrothermal conversion elements and a method by which medicine is ejected from the ejection openings using a pressure oscillation from an electromechanical conversion element (such as a piezoelectric element) which confers mechanical energy to the medicine. The ejection method can be selected according to the type of medicine.

When the thermal jet method is used, it is possible to achieve a high accuracy and reproducibility in the diameter of the ejection openings, the amount of heat of the thermal pulse used for the ejection, and in the microheaters used as the electrothermal conversion elements for each of the liquid ejecting units. As a result, a narrow droplet diameter distribution can be achieved. Also, since the manufacturing cost of the head is low, the thermal jet method is readily applicable to small apparatuses in which the head has to be frequently exchanged. Hence, when portability and convenience are necessary in the medicine ejection apparatus, the ejection apparatus of the thermal jet method may be preferable.

The medicine cartridge 10 is also provided with an authentication unit. The cartridge authentication unit makes use of a well-known authentication unit such as a bar code, a QR code, an RFID, and an IC chip. A reading unit for performing the authentication can use an image-based, an electrical, or a radio-wave based identification method. Specifically, a CCD, a CMOS, an electrical connection, or an antenna can be illustrated.

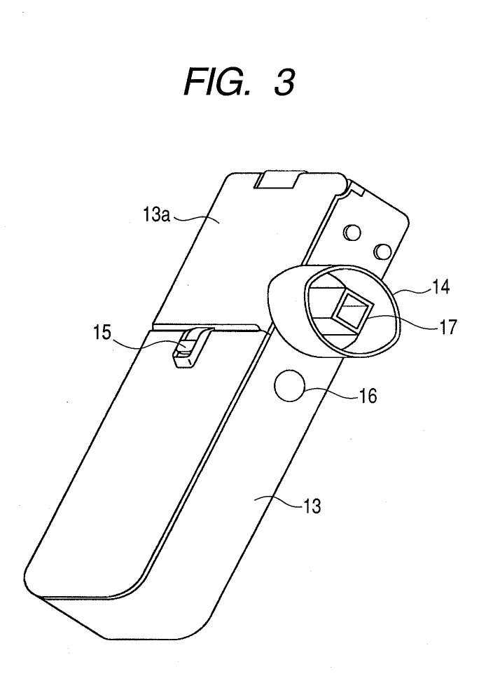

FIG. 3 is a perspective drawing illustrating an appearance of the inhaler 1. An inhaler body includes a housing 13 storing a plurality of medicine cartridges 10, a controller for the medicine cartridges 10, a power source (battery). A mouthpiece 14 used for measuring lung function and for inhaling is installed in the housing 13.

A claw-form provided on a tip of a lock lever 15 is urged towards a protrusion provided at a front edge of the access cover 13a by a spring. The claw-form is formed so as to catch on the protrusion and thereby prevent the access cover 13a of the housing 13 from opening during use. When the lock lever 15 is slid downwards, a force from an access cover returning spring which urges the access cover 13a causes the access cover 13a to rotate and open.

The medicine cartridge 10 is constructed with the reservoir 10a and the head portion 11 integrated as shown in FIG. 2, and so as to be exchangeable when the access cover 13a is opened.

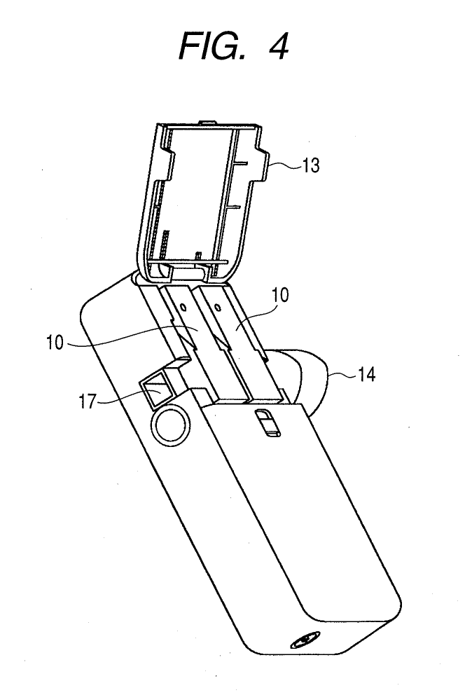

FIG. 4 is a perspective drawing illustrating an appearance of the inhaler 1 with the access cover 13a in an open state. The plurality of medicine cartridges 10 are provided part-way along a pipe-form air path which leads inflowing air from air intake openings into the air flow path 17. At the head portion 11 of each medicine cartridge 10, the medicine is sprayed to form fine particles and mixed with the air flowing in the pipe-form air flow paths.

The inhaler 1 makes use of a method by which the user holds the mouthpiece 14 between their lips, and inhales, causing air to flow into the air inlet. In other words, the inlet portion is constructed as an inhaling mechanism which causes the target of the medication (the user) to inhale a gas having floating therein fine particles of medicine generated by the spraying mechanism.

A method by which the beginning of the ejection is synchronized with the user inhaling may be used. Alternatively, a method by which the user decides when the operations should begin via a button may be used.

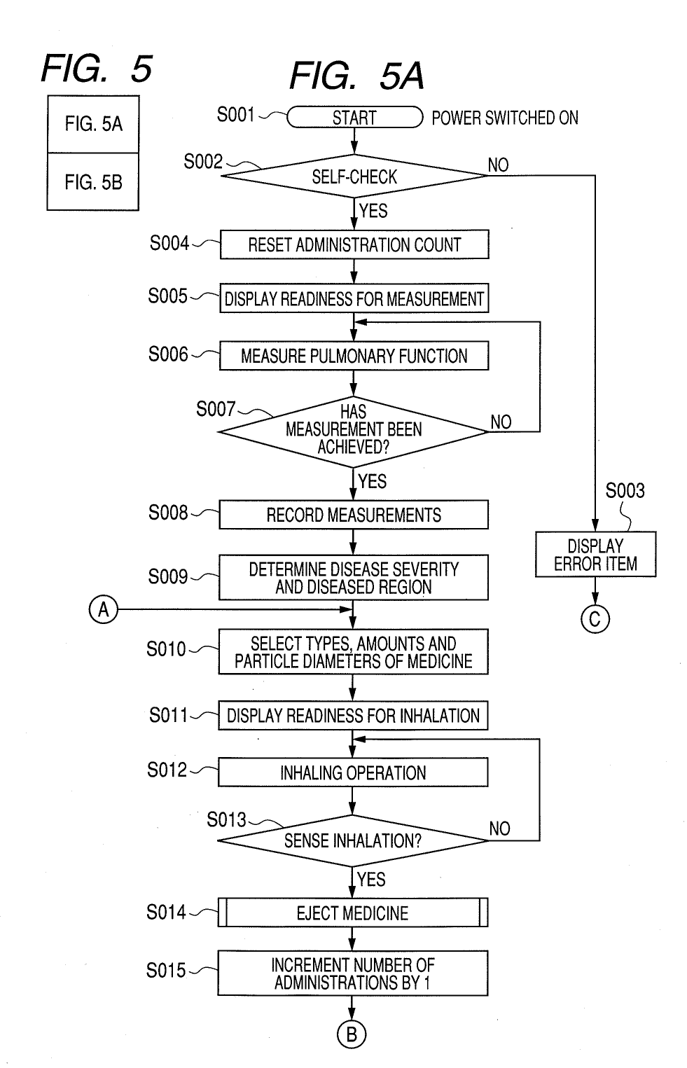

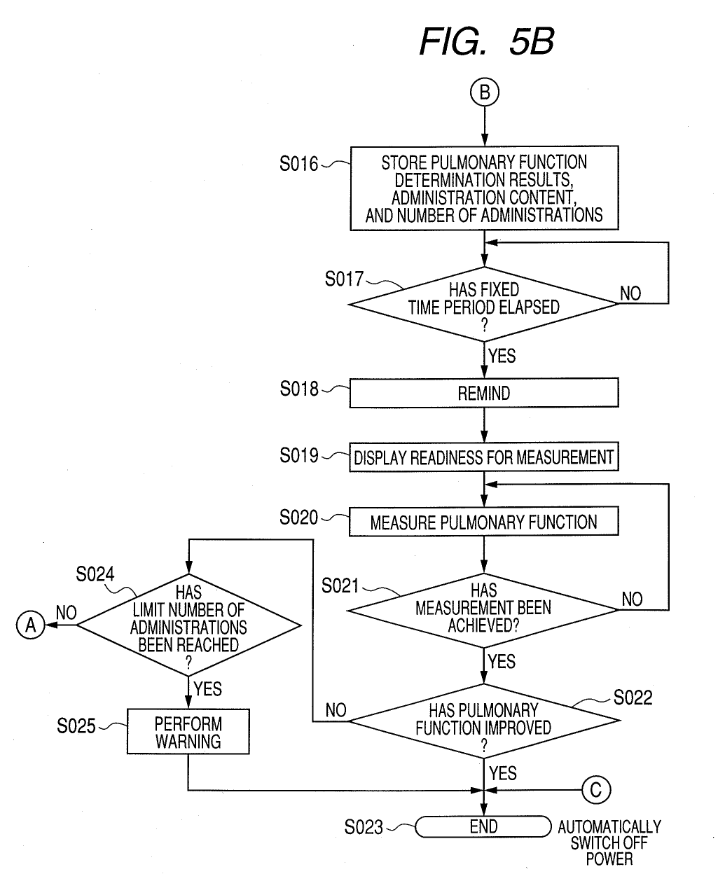

FIG. 5A and FIG. 5B illustrate a flow of operations in the inhaler 1. First, the processing is caused to enter a start-of-use state by the user performing an operation such as pressing the power button 16 (step S001). Next, self-checks are performed to check, for instance, whether a plurality of medicine cartridges 10, each of which is a single body made up of the reservoir 10a for storing the medicine and the head portion 11 for ejecting the medicine, are present in the inhaler 1, and to self-check an amount of charge remaining in the battery (step S002). When the ejection is performed using the thermal ink jet method, the detection of the presence of the medicine cartridges 10 can be realized by, for instance, measuring resistance values of the heaters which form the ejection energy generation unit. When medicine cartridge 10 cannot be detected or the remaining charge on the battery is insufficient, the user is notified by display of a message indicating that the medicine cartridges 10 should be reinstalled or that the battery should be recharged (step S003), and the power is switched off (step S023). On the other hand, when the self-checks indicate that there is no problem, which is to say when the medicine cartridges 10 have been detected and sufficient charge remains on the battery, the processing resets the number of administrations (step S004), and displays a message indicating readiness for lung function measurements (step S005).

The processing measures the lung function of the user using the spirometer function included in the lung function measuring unit 2 (step S006), and checks to confirm that the measurements have taken place (step S007). When the measurements have not taken place, the processing notifies the user by displaying a message indicating that the measurements should be repeated, and repeats the lung function measurements. On the other hand, when the measurements have taken place, the processing records the results (step S008). The processing determines the disease severity and the diseased region from the lung function measurements (step S009), and selects a medicine type, amount, and particle diameter appropriate for the determined lung condition (step S010). The determination of the lung condition is performed by comparing the lung function measurements with information in a table (medication pattern table) which has been stored in advance in the storage unit 7.

The table used to determine the lung condition includes at least one of FEV1 and PEF values obtained from the lung function measurements, disease severities determined from the measurements, and airway obstruction patterns determined from the pattern of a flow volume curve (flow volume pattern). The flow volume pattern and the corresponding airway obstruction regions are shown in Table 1. As shown in the table, the airway obstruction region is determined from the presence or otherwise of a peak disappearance, which is an indicator of an upper airway obstruction, and from a V50/V25 value (ratio of V50 and V25) which is an indicator of a lower airway obstruction.

TABLE 1

| | | Flow Volume Pattern | |
|---|---|---|---|
| | | Peak Disappearance | V50/V25 |
| | obstruction | (Indicator of | (Indicator of |
| | Region | Upper obstruction) | Lower obstruction) |
| A | Upper Airway | Peak Disappearance | Less than 3 |
| B | Lower Airway | No Peak Disappearance | 3 or more |
| C | Upper and | Peak Disappearance | 3 or more |
| | Lower Airways | | |

Here, V50 is the air flow rate at an air volume that is 50% of vital capacity, and V25 is the air flow rate at an air volume that is 25% of vital capacity. Further, the values for FEV1 and PEF, the disease severities, and the flow volume patterns and obstruction regions are recorded in one-to-one correspondence with types, particle diameters, and amounts of the medicine to be ejected.

When the types, amounts, and particle diameters of the medicine to be ejected have been selected, the processing displays that the inhaler is ready (step S011). Note that readiness may alternatively be indicated using an LED. After seeing the signal indicating that inhaler preparation is complete, the user starts an inhaling operation (step S012). When an inhalation is sensed (step S013), the processing displays notification to show the user that ejection is taking place and ejects the medicine from the respective medicine cartridges 10 (step S014). The sensing of the inhalation is performed by a sensor capable of measuring air flow, such as a pressure sensor provided in communication with the air duct formed in the mouthpiece 14. The pressure sensor senses a drop in pressure in the flow path resulting from the inhalation by the user. The processing then increments the number of administrations (step S015), and stores the measurements of the disease severity and diseased region, the medication content, and the number of administrations (step S016). A fixed period, for example 30 minutes, after the inhalation of the medication has elapsed (step S017), the processing reminds the user to repeat the measurement of the lung function using an alarm (step S018), and performs display to indicate readiness for the lung function measurements. The processing then measures lung function (step S020), and checks whether the measurements have taken place (step S021).

When the measurements have taken place, the processing stores the lung function measurements and compares the stored measurements with the lung function measurements taken (in step 5008) before administering the medicine to check whether lung function has improved in comparison to before administering the medicine (step S022). For instance, when COPD has been diagnosed in the user, the processing compares FEV1 values from before and after administering the medicine, and determines that there has been an improvement in lung function if increases of at least 200 mL and at least 12% are seen. When no improvement is seen in the lung function after administering the medication, the processing checks whether the limit on the number of administrations has been reached (step S024). When the limit has not been reached, the processing returns to the step (step 5010) for selecting the medication content. When the number of administrations reaches the limit number without any improvement in lung function, a warning suggesting that the user consults a doctor is issued (step S025), the power is switched off, and the processing ends (step S023). The limit number of administrations is decided based on factors such as the body-type of the user, the characteristics of the medicine, and the lung function and symptoms of the user. When determining that there has been an improvement in lung function in the check for improvement (step S022), the power is switched off and the processing ends (step S023).

The inhaler of the present invention measures lung function, determines from the measurements types, amounts, and particle diameters of the medicine to be ejected, and ejects the selected medicine. The inhaler measures lung function using a spirometer, determines disease severity from FEV1 (forced expiratory volume in one second) and PEF (peak expiratory flow) values obtained from the measurements, and determines the diseased region of the lungs from the pattern of the flow volume curve. Types and amounts of medicine are selected based on the severity and a particle diameter is selected based on the diseased region. The medicine required for the treatment is then ejected in a single use. For instance, in the case of bronchial asthma, when it is determined that the severity is mild using the FEV1 and PEF values and that the obstruction is in the small airways from the flow volume curve, it is preferable that the user should inhale low doses of a bronchodilator with a particle diameter of 2 μm to 3 μm and inhaled steroids with particle diameters of 2 μm to 3 μm and 5 μm to 7 μm. To realize such an inhalation, a driving method for the inhaler includes steps of measuring lung function, determining the types, amounts, and particle diameters of the medicine to be ejected using the measurements, and controlling the ejection so that medicine suitable for the lung condition is used. The inhaler of the present invention driven by the above-described method is constructed to measure lung function, and control an ejection unit so as to eject medicine that has been selected based on the measurements.

The inhaler of the present invention can include a plurality of applied portions for cartridges (medicine cartridge) each having at least a reservoir containing a medicine. Hence, when medicine cartridges are attached to the plurality of applied portions, medicines of a plurality of types can be administered. The lung function measurements may, in some cases, indicate that simultaneously administering a plurality of medicines is preferable, and the inhaler has the flexibility to support such cases. The medicine ejection portion may be integrated with the reservoir in the cartridge, or may be provided in the inhaler. When provided in the inhaler, a plurality of ejection portions may be provided so as to correspond with the cartridges. Alternatively, a single ejection portion may be provided. The number of cartridges can be freely set according to the number of medicines to be simultaneously inhaled.

For instance, three cartridges A, B, and C, may be provided with a medicine a in the reservoir of cartridge A, a medicine b in the reservoir of cartridge B, and a medicine c in the reservoir of the cartridge C. Depending on the disease severity determined from the lung function measurements, the inhaler can be set to eject the medicine a alone, or all of the medicines, a, b and c. Moreover, depending on the diseased region determined from the lung function measurements, the inhaler can be set to eject the medicine a with two differing particle diameters. Moreover, the medicine a and the medicine b can be ejected with differing particle diameters.

In the present invention, the medicine is not limited to being a pharmacological compound which shows pharmacological and physiological effects, and may include a flavoring or scenting component, a dye, or a pigment. Moreover, the medicine may be in liquid or powder form.

The liquid medicine in the present invention refers to a medicine that is a liquid or to a liquid medium having a medicine uniformly distributed therein. Any materials which can be uniformly distributed in the liquid are acceptable as constituents of the liquid. The uniform state in the liquid can be achieved using any one of a solution, a dispersion, an emulsion, a suspension, and a slurry.

When a liquid medicine is used as the medicine, the principle medium of the liquid can be water or an organic compound, but, given that the liquid medicine is to be administered to a living body, is preferably water.

The above-mentioned pharmacological compounds which bring about physiological effects can be any of various widely used medical compounds. Specific examples include bronchodilators such as β₂-agonists and anticholinergics, glucocorticosteroids, non-steroid anti-inflammatory drugs, Theophyllines, anti-asthmatic drugs, anti-allergy drugs, antagonists, expectorants, antitussives, and sedatives. Further examples include depression-treating drugs, analgesics, mast-cell stabilizers, anti-histamines, antiemetics, sleep-inducing drugs, vitamins, sex steroid hormones, anti-tumor agents, anti-arrhythmic drugs, anti-hypertensive drugs, anti-anxiety drugs, anti-psychotic drugs, cardiotonics, and drugs to aid smoking cessation. Further examples include obesity-treating drugs, migraine drugs, anti-rheumatic drugs, protein therapeutics, hormone drugs, cytokines, receptors, antibodies, enzymes, enzyme inhibitors, vaccines, antisenses, genes, DNA and RNA.

Various natural essences, synthetic essences, and mixed essences can be used as the above-described flavoring or scenting components. It is also possible to use a general-purpose essence component used in cosmetic perfumes, soap perfumes, and food product flavorings. The added secondary components are preferably multi-purpose pharmaceutical additives recorded in various National Pharmacopoeia or additives approved for use in food products and cosmetic products.

The content ratio of the essence or the like included as the flavoring component or the scenting component differs depending on the essence. Generally speaking, however, the content is preferably in a range of 1 ppb to 10% and more preferably in a range of 1 ppb to 1%. Alternatively, a combination of flavoring component and scenting component may be used in quantities which do not adversely affect the intended use of the ejection liquid.

Various dyes and pigments can be used as the above-mentioned dyes and pigments. The added secondary components are preferably multi-purpose pharmaceutical additives recorded in various National Pharmacopoeia or additives approved for use in food products and cosmetic products.

The content of a colorant included as the above-mentioned dye or pigment differs depending on the colorant. Generally speaking, however, the content is preferably in a range of 1 ppm to 30% and more preferably in a range of 0.01% to 10%. Alternatively, a combination of dyes and pigments may be used in quantities which do not adversely affect the intended use of the ejection liquid.

Where necessary, an ejection aiding agent, an absorption accelerant, or an absorption inhibitor can be used. The medicine may be a hydrophobic material in which the essence or colorant does not exhibit a desired solubility. In such a case, a dispersant or a surfactant for achieving a uniform distribution can be added as necessary. Moreover, various other additives suitable for the indented use of the spray can be added as required. Suitable additives may include dispersants, surfactants, surface control agents, viscosity control agents, solvents, wetting agents, and pH controlling agents.

Mixable additives include ionic surfactants, non-ionic surfactants, emulsifiers, dispersants, hydrophilic binders, hydrophobic binders, hydrophilic thickening agents, hydrophobic thickening agents, glycerines, glycols, and glycol derivatives. Further examples include alcohols, amino acids, ureas, electrolytes, and buffer solutions. Note that a single one or a plurality of the additives can be added as required.

With regard to materials used as the above-described example additives, the added secondary components are preferably multi-purpose pharmaceutical additives recorded in various National Pharmacopoeia or additives approved for use in food products and cosmetic products.

The respective contents (mass concentrations) of the various materials included as the above-described additives differ depending on the medicinal compounds which form the principle component of the medicine, the type of essence used as the flavoring or scenting component, and the type and content of the colorant. However, the contents can be defined as follows. Generally, the content of the one or more additives is preferably in a range of 0.01 mass % to 40 mass % and more preferably in a range of 0.1 mass % to 20 mass %. Alternatively the amounts of the above-described additives can be set according to the additive application (function), type, and combination. For ease of ejection of the prepared medicine, when the total content of the medicine of the liquid medicine, the flavoring or scenting component, and the colorant is 1 part by mass, the content of the other additives is preferably selected to be in a range of 0.5 parts by mass to 100 parts by mass.

The medicines filling the plurality of reservoirs are selected from the above. The medicines in all the reservoirs may be identical, but preferably differ from reservoir to reservoir. For instance, differing medicines can be used in each reservoir or a medicine and a surfactant can be used. The composition held in each reservoir may be a mixture of a medicine, colorant, or an essence with additives, or a mixture of materials selected from medicines, essences, and colorants.

The inhaler of the present invention can store lung function measurements, disease severity, diseased regions, and information about the types, amounts and particle diameters of the medicines to be ejected. Moreover, the inhaler can store times at which the lung function has been measured and times at which the medicine has been ejected. This information is stored automatically, and the stored content can be viewed at any time.

The determination of the types, amounts and particle diameters of the medicine from the lung function measurements is not limited to medicine that is to be ejected immediately after the lung function measurements. For instance, the lung function measurement can be used as feedback even when the medicine is to be ejected a day after the lung function has been measured. When a given drug has been inhaled over a fixed period and an improvement has been seen, or not seen, the type or amount of the drug to be ejected at the next and in subsequent uses of the inhaler can be altered. When no improvement in lung function is seen after the medicine has been inhaled, the measured lung function can be compared with the lung function before administering the medicine, and more of the same medicine can be added and ejected. Alternatively, the medicine can be supplemented with a different type of medicine.

The inhaler can include a display unit capable of displaying the process from the lung function measurements to the determination of the medication to be ejected. The display unit displays the lung function measurements, the disease severity and the diseased region determined from the results, and the selected types, amounts and particle diameters, enabling the user of the inhaler to continue checking the process right up to the ejection of medicine.

The inhaler of the present invention can include an alarm function for notifying the user of the times at which to measure the lung function. Although continuous measurements of lung function are preferably made before and after each inhalation of the medicine, it is conceivable that the user will forget to make a measurement. For instance, it may be preferable to make a measurement 30 to 60 minutes after the inhalation of the medicine. The alarm would then alert the user that the predetermined time period has elapsed since the ejection of the medicine, and a further measurement of the lung function is made.

When there is a marked drop in lung function or an acute attack occurs, the inhaler can warn the user to consult a doctor.

The user can be distinguished using a personal identification function. Information identifying the user is input, and the inhaler calls up previous lung function measurements and administration records based on the information.

It is possible to attach mouthpieces of differing forms when measuring the lung function and inhaling the medicine. This allows the mouthpieces best suited for the lung function measurements and the inhalation to be selected.

The control of the timing for the starting and stopping of the ejection of the medicine can be performed by controlling the driving of the ejection portion in a desired manner via electronic control using a program. Since it is possible to control the amount of medicine to be ejected and precisely control the timing of the ejection, a high reproducibility can be achieved in the ejection.

Since the inhaler of the present invention can be employed with the plurality of medicines remaining a separated state, there is no need to consider a stability of the storage (known as “pot life”).

It is also possible to increase the amount of medicine that is ejected by storing identical medicines in a plurality of the reservoirs.

It is possible to use medicine ejection portions (ejection portions) having nozzle diameters which can be freely set. When the above-described medicine is used for inhalation, the particle diameter of the prescribed material of the present invention is preferably from 0.5 μm to 20 μm and with a narrow particle diameter distribution.

When the liquid medicine is to be sprayed, the particle diameter is controlled by the nozzle diameter. When a powder is to be sprayed, the medicine is dried by a known method to achieve the desired particle diameter.

As a result, it is possible to change the region in the lungs to which the medicine is delivered, and deliver suitable medicine to the region determined to be the diseased region based on the lung function measurements. To deliver the medicine to the alveoli and the small airways, the particle diameter is preferably from 2 μm to 3 μm. For the central and upper airways, the particle diameter is preferably from 5 μm to 7 μm.

By using the above-described inhaler to measure lung function and ejecting medicine of types, amounts, and particle diameters dependent on the measurements, it is possible to maximize the effects of the medicine by intaking them.

### Example 1

The inhaler illustrated in FIGS. 1 to 4 was used to measure the lung function of a user in whom bronchial asthma had been observed, and medicine was ejected based on the measured lung function. The medicine cartridges held Salbutamol powder (a short-acting β₂-agonist), Salmeterol powder (a long-acting β₂-agonist), and Fluticasone powder (an inhaled glucocorticosteroid). When the lung function of the user was measured, the FEV1 value was 85% of the predicted value with a change of 15%, the V50/V25 value on the flow volume curve was 3 or more, and a disappearance of the peak was not observed. Based on these lung function measurements, the bronchial asthma severity was determined to be mild to intermittent, and the occluded region was determined to be the lower airways. Based on these results, Salbutamol (particle diameter of 2 μm to 3 μm, dose of 200 μg) and a Fluticasone (particle diameters of 2 μm to 3 μm and 5 μm to 7 μm and a dose of 25 μg) were selected as the medicines to be ejected. The selected medicines were then ejected.

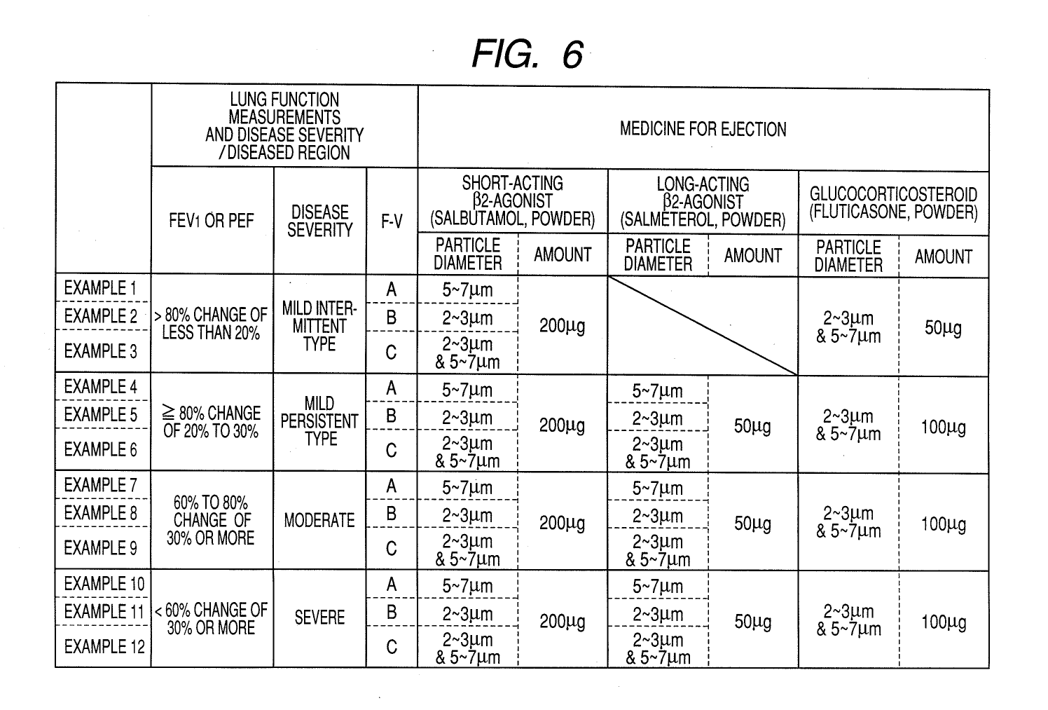

Lung function measurements and medicine ejection patterns for the cases of adult bronchial asthma in the present examples are shown in FIG. 6. In Examples 13 to 21, the medicine cartridges held Ipratropium in aqueous solution (a short-acting anticholinergic), Tiotropium powder (a long-acting anticholinergic), and Beclomethasone powder (an inhaled glucocorticosteroid). In Example 22 to 30, the medicine cartridges held Fenoterol in aqueous solution (a short-acting β₂-agonist), Salmeterol powder (a long-acting β₂-agonist), and Budesonide powder (an inhaled glucocorticosteroid).

### Examples 2 to 30

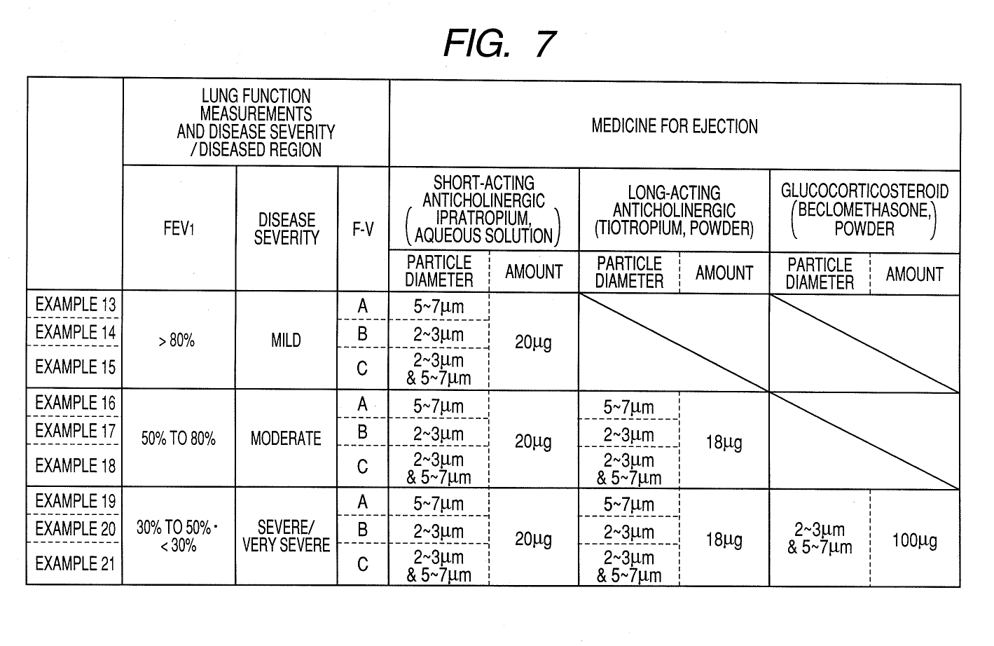

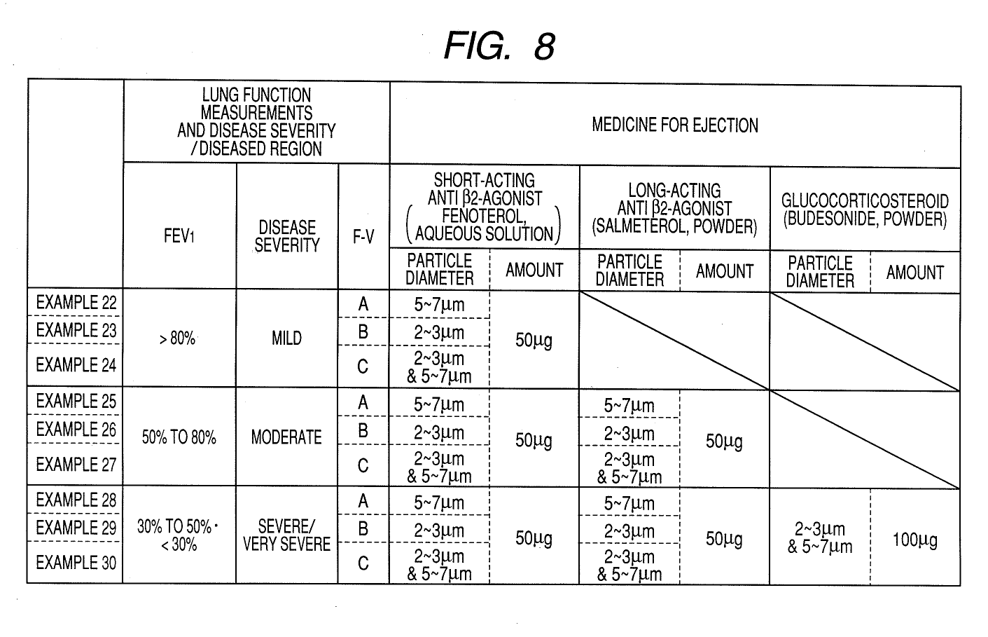

Like Example 1, Examples 2 to 12 are cases in which adult bronchial asthma has been observed in the users. The lung function measurements and medicine ejection patterns are illustrated in the table of FIG. 6. Examples 13 to 30 are cases in which medicine is ejected after measuring the lung function of users in whom chronic obstructive pulmonary disease (COPD) has been observed. The lung function measurements and medicine ejection patterns are illustrated in the tables of FIG. 7 and FIG. 8.

### Example 31

In Examples 1 to 30, the short-acting and long-acting bronchodilators are both β₂-agonists or both anticholinergics, but it may be the case that one bronchodilator is a β₂-agonists and the other is an anticholinergic.

The medicine cartridges store Fenoterol in aqueous solution (a short-acting β₂-agonist), Tiotropium powder (a long-acting anticholinergic), and Beclomethasone powder (an inhaled glucocorticosteroid). The lung function of user in whom chronic pulmonary obstructive disease (COPD) had been bereaved was measured, and medicine dependent on the measured lung function was ejected. When the lung function of the users was measured, the FEV1 value was 60% of the predicted value, the V50/V25 value on the flow volume curve was 3 or more, and a disappearance of the peak was observed. Based on the lung function measurements, the severity of the COPD was determined to be medium, and the occluded region was determined to be the upper airways and the lower airways. Based on these results, Fenoterol (with particle diameters of 2 μm to 3 μm and 5 μm to 7 μm, and respective doses 25 μg from 10 μL aqueous solutions at a concentrations of 2.5 mg/mL) and a Tiotropium (with particle diameters of 2 μm to 3 μm and 5 μm to 7 μm and respective doses of 9 μg) were selected as the medicines to be ejected, and the selected medicines were ejected.

### Example 32

Using the inhaler illustrated in FIGS. 1 to 4, it is possible to measure the lung function of a user before and after the medicine is inhaled, and supplement the medicine when an improvement in lung function is not observed after the inhalation.

When the lung function of a user having the same lung function as in Example 31 was measured 30 minutes after the inhalation of the medicine, the FEV1 was 60% of the predicted value, and no improvement over the lung function before the inhalation was observed. From these results, it was determined that no improvement in lung function was observed, and supplementary Fenoterol (with particle diameters of 2 μm to 3 μm and 5 μm to 7 μm, and respective doses of 25 μg from 10 μL aqueous solutions at a concentration of 2.5 mg/mL) and Tiotropium (with particle diameters of 2 μm to 3 μm and 5 μm to 7 μm and respective doses of 9 μg) were ejected. When the lung function was measured 30 minutes after the inhalation of medicine, the FEV1 value was 80% of the predicted value and had increased by 200 mL, and it was therefore determined that an improvement in lung function had occurred after the supplementary medication. From these results, the medication content corresponding to the 50% to 60% of the predicted FEV1 value was selected using the next lung function measurements. In other words, when the V50/V25 value on the flow volume curve was 3 or more, and a disappearance of the peak was observed, Fenoterol (with particle diameters of 2 μm to 3 μm and 5 μm to 7 μm, and respective doses of 50 μg from 10 μL aqueous solutions at concentrations of 5.0 mg/mL) and Tiotropium (with particle diameters of 2 μm to 3 μm and 5 μm to 7 μm and respective doses of 18 μg) were selected as the medicine to be ejected.

The present invention is not limited to the above embodiments and various changes and modifications can be made within the spirit and scope of the present invention. Therefore to apprise the public of the scope of the present invention, the following claims are made.

This application claims priority from Japanese Patent Application No. 2007-148844 filed on Jun. 5, 2007, which is incorporated hereinto by reference.

1. An inhaler for causing a user to inhale ejected medicine, comprising:
a lung function measuring unit operable to measure lung function;
a computing unit operable to find a type and a particle diameter of medicine suitable for a disease severity and a diseased region determined from measurements by the lung function measuring unit; and
an ejection unit operable to eject the medicine based on output from the computing unit.

2. The inhaler according to claim 1, further comprising a plurality of applied portions for medicine cartridges which store the medicine, wherein when a plurality of medicine cartridges are attached to the plurality of applied portions, a plurality of medicine types can be administered.

3. The inhaler according to claim 1, wherein a disease severity is determined from an FEV1 (forced expiratory volume in one second) or a PEF (peak expiratory flow) among the measurements of lung function by the lung function measuring unit, and a diseased region is determined from a flow volume pattern among the measurements of lung function by the lung function measuring unit.

4. The inhaler according to claim 1, further comprising:
a recording unit operable to record the measurements by the lung function measuring unit and a medication content ejected by the ejection unit,
wherein lung function is measured again after administration of the medicine, resulting measurements are recorded and compared with the measurements from before administering the medicine, and supplementary medicine and a subsequent medication pattern are selected based on the comparison.

5. A driving method for an inhaler for causing a user to inhale ejected medicine, comprising:
measuring lung function of the user using a lung function measuring unit;
recording measurements by the lung function measuring unit;
comparing the measurement made by the lung function measuring unit with a medication pattern table, and selecting a type and a particle diameter of the medicine to be administered;
ejecting the medicine of the selected type and particle diameter from an ejection unit; and
recording the medication content for the ejection by the ejection unit in a recording unit.

6. An inhaler for causing a user to inhale ejected medicine, comprising:
a lung function measuring unit operable to measure lung function;
a computing unit operable to find a type of medicine suitable for a disease severity determined from measurements by the lung function measuring unit; and
an ejection unit operable to eject the medicine based on output from the computing unit.
